(19) United States
(12) Patent Application Publication
(10) Pub. No.: US 2023/0277825 A1
(43) Pub. Date: Sep. 7, 2023

(54) MRNA VACCINE MICRONEEDLE PRODUCT AS WELL AS PREPARATION METHOD AND APPLICATION THEREOF

(71) Applicants: Healthina Stem Cell Industry Platform (Tianjin) Limited, Tianjin (CN); Tangyi Holdings(Shenzhen) Limited, Shenzhen (CN)

(72) Inventors: Bin ZHENG, Tianjin (CN); Yulin CAO, Beijing (CN); Wei SUN, Tianjin (CN); Shixiang CHENG, Tianjin (CN)

(73) Assignees: Healthina Stem Cell Industry Platform (Tianjin) Limited, Tianjin (CN); Tangyi Holdings (Shenzhen) Limited, Shenzhen (CN)

(21) Appl. No.: 17/743,477

(22) Filed: May 13, 2022

(30) Foreign Application Priority Data
Mar. 2, 2022 (CN) 202210198247.3

(51) Int. Cl. A61M 37/00; A61L 31/14; A61L 31/16

(52) U.S. Cl. CPC A61M 37/0015; A61L 31/148; A61L 31/16; A61M 2037/0046; A61M 2037/0053

(57) ABSTRACT

An mRNA vaccine microneedle product includes a backing and a mRNA vaccine solution-containing microneedle array attached to a side of the backing, wherein the mRNA solution-containing microneedle array includes several microneedles, and each microneedle contains a matrix and mRNA loaded in the matrix. The present invention adopts the mRNA vaccine microneedle product as well as a preparation method and an application thereof, and such microneedle product can realize a rapid humoral immunity after being administered to the skins of animal bodies (especially human bodies), which solves the problems of traditional vaccination, such as muscular pain and multiple injections.

### CROSS REFERENCE TO THE RELATED APPLICATIONS

This application is based upon and claims priority to Chinese Patent Application No. 202210198247.3, filed on Mar. 02, 2022, the entire contents of which are incorporated herein by reference.

### TECHNICAL FIELD

The present invention relates to the technical field of biological microneedles, in particular to a mRNA vaccine microneedle product as well as a preparation method and an application thereof.

### BACKGROUND

At present, the epidemic around the world is still very serious, and has resulted in immeasurable losses to the economy and people’s lives of all countries. As the epidemic is gradually controlled, widespread vaccination plays a relatively important role. Current pneumonia vaccines include inactivated vaccines, recombinant subunit vaccines, adenovirus vector vaccines and nucleic acid vaccines, which are mainly administrated by intramuscular injection.

However, the intramuscular injection presents some insurmountable problems. For example, cold chain storage is required, and cold chain storage greatly increases the costs of transportation, storage and use; the intramuscular injection requires a certain injection dose to take effect, generally one or two or more injections are administrated, and both the use of medical resources and the time arrangement of vaccinees are problematic.

Served as a new technique of locally transdermal administration, microneedle administration combines the convenience of emplastrum and the effectiveness of subcutaneous injection, avoids the shortcomings of other administration methods, and features the advantages of no nerve access, safety, no pain, efficient penetration, etc.

A microneedle product generally includes a plurality of microneedles with a length of no more than 1 mm. The microneedles can form micro channels in the skin cuticle, break through the barrier of the skin cuticle and promote the penetration of drugs, thus reducing a dose of drugs accumulated in the cuticle and increasing a dose of drugs reaching the epidermal, dermal and subcutaneous tissues. Therefore, the microneedle products are widely used to promote the transdermal absorption of small-molecule and large-molecule drugs, and to treat many diseases such as obesity, diabetes and cancers, with a wide application prospect. Moreover, the microneedle products are extremely convenient to use, require no professional training, allow patients to be administered by themselves, pose a low risk of accidental needle injury, and are easy to dispose after use. However, it is difficult to store vaccines in microneedle patches.

### SUMMARY

The present invention is intended to provide a mRNA vaccine microneedle product as well as a preparation method and an application thereof, wherein such microneedle product can realize a rapid immune response by loading a mRNA-containing solution in the microneedle product after being administered to the skin, and a long-acting stable release and rapid response of a vaccine can be achieved.

To achieve the aforesaid purposes, the present invention provides a mRNA vaccine microneedle product, including a backing and a mRNA vaccine solution-containing microneedle array attached to a side of the backing, wherein the mRNA solution-containing microneedle array includes several microneedles, wherein each microneedle contains a matrix and mRNA loaded in the matrix.

Preferably, each of the microneedles includes a tip and a bottom, wherein the tip is away from the backing, the bottom is attached to the backing, a vertical distance from the tip to the bottom is 200 µm-1 mm, a diameter of the bottom is 100 µm-500 µm, and a spacing between the adjacent tips is 300 µm-800 µm.

Preferably, the matrix material is one or more of polyethylene glycol diacrylate, silk fibroin, gelatin, hyaluronic acid, polylactic acid-glycolic acid copolymer, polylactic acid, polyvinylpyrrolidone (PVP) and polyvinyl alcohol, preferably gelatin and/or hyaluronic acid, and more preferably gelatin, and the matrixes are formed by crosslinking and/or drying and curing of their own aqueous solution.

Preferably, the backing material is one or more of polyethylene glycol diacrylate, silk fibroin, methacrylate gelatin (GelMA), carboxymethyl cellulose, trehalose, hyaluronic acid, polylactic acid-glycolic acid copolymer, polylactic acid, galactose, PVP and polyvinyl alcohol, and the backing is formed by crosslinking and/or drying and curing of its own aqueous solution.

A method for preparing the mRNA vaccine microneedle product, including the following steps:
S1, preparing mRNA;
S2, adding a matrix material forming a matrix to the mRNA-containing liquid medium obtained in S1 to form a mixed solution;
S3, placing the mixed solution obtained in S2 in molding holes of a microneedle mold, and filling at least a portion of the volume of the molding holes, and preferably the whole volume of the molding holes;
S4, crosslinking the mixed solution containing the matrix material and the mRNA in the molding holes, and preferably performing ultraviolet crosslinking for 10s, and/or performing drying and curing, and preferably performing hot curing, to form microneedles in the molding holes;
S5, applying a backing material-containing solution to bottom surfaces of the microneedles and an upper surface of the microneedle mold that is not covered by the microneedles to form a backing solution layer, and crosslinking the backing material to form a continuous backing layer, so that the mRNA vaccine microneedle array is attached to the backing solution layer or the backing layer; and
S6, drying and curing the backing solution layer or the backing layer obtained in S5 and the mRNA vaccine microneedle array to form a mRNA vaccine microneedle product.

Preferably, the method for preparing the mRNA includes the following steps:
(1) constructing plasmids which contain designed sequence modules, including spike protein coding; breaking cell membranes by electric current, and introducing circular DNA plasmids into Escherichia coli;
(2) storing the Escherichia coli in a solution containing a large amount of nutrients and performing reproduction and amplification to obtain trillions of DNA plasmids;
(3) extracting and purifying the DNA plasmids, and filtrating the solution to remove bacteria and other substances;
(4) cleaving the purified circular DNA plasmids into strands by enzyme;
(5) transforming the DNA strands into mRNA: mixing the DNA strands obtained in the previous step with enzyme and nucleotide, so that the RNA polymerase transcribes DNA into mRNA;
(6) encapsulating the mRNA into a lipid carrier, wherein the lipid is suspended in an alcoholic solution, comes into contact with the mRNA and encapsulates the mRNA, and such two substances are attracted by opposite charges; and
(7) filtering excess impurities from the stock solution through tangential flow filtration (TFF), and preparing a final mRNA vaccine solution.

Preferably, in S2, a concentration of the matrix material in the mixed solution is 20%-40% of a total weight of the mixed solution, and preferably 30%.

Preferably, in S2, a microneedle mold is provided, and such microneedle mold includes an upper surface and molding holes extending downward from the upper surface, wherein each of the molding holes includes a tip and a bottom, the tip is away from the upper surface, the bottom plane is flush with the upper surface, a distance from the tip to the bottom is 200 µm-1 mm, a diameter of the bottom is 100 µm-500 µm, and a spacing between the adjacent tips is 300 µm-800 µm.

Preferably, the backing material is hyaluronic acid, and the matrix material is GelMA.

The present invention further provides an application of a mRNA vaccine microneedle product and a preparation method thereof in preparing medical devices against pneumonia-associated viruses.

The present invention further provides an application of a mRNA vaccine microneedle and a preparation method thereof in preparing drugs against pneumonia-associated viruses.

Therefore, the present invention adopts the mRNA vaccine microneedle product as well as a preparation method and an application thereof, wherein the microneedle product can achieve a rapid therapeutic effect by loading a synthetic RNA protein in the microneedle product after being administered to the skin, and such RNA has the functions of neutralizing a specific virus and invalidating a special key for the specific virus to invade a human body. A microneedle patch can reduce a muscular adverse reaction of injection, and can be released in a long time. A vaccine present in the microneedle patch can not only work for a long time, but also reduce the discomfort caused by intramuscular injection and the use of medical resources.

Specifically, the present invention features the following advantages:

1. According to the mRNA vaccine microneedle product of the present invention, a RNA protein is artificially synthesized and injected into a human body. Such RNA can then be used as a template to make a characteristic protein designed in the laboratory, following normal biological mechanisms. Such RNA is usually designed in the laboratory according to a certain spike characteristic of a virus. Therefore, a specific protein made by such RNA as a template has the functions of neutralizing a specific virus and invalidating a special key for the specific virus to invade a human body.

2. The mRNA vaccine microneedle product of the present invention can pierce the skin cuticle which limits drug absorption, and promote the spread of mRNA into a human body without causing pain.

3. Microneedles are prepared by a microneedle template reverse mold. The method is simple, easy to operate, low-cost, reusable, easy to control the basic shape of a microneedle array, highly safe, and suitable for promotion, without needing high technical requirements.

The technical solutions of the present invention will be further described below in detail in combination with the accompanying drawings and embodiments.

### BRIEF DESCRIPTION OF THE DRAWINGS

FIG. 1 is a structural side view of a part of a mRNA vaccine microneedle product of the present invention.

FIG. 2 is a structural side view of a part of a microneedle mold for preparing a mRNA vaccine microneedle product of the present invention.

Labels in the figures:

100. mRNA vaccine microneedle product; 110. microneedle; 120. backing; 200. microneedle mold; 201. molding hole; 202. upper surface.

### DETAILED DESCRIPTION OF THE PREFERRED EMBODIMENTS

The technical solutions of the present invention will be further described below in combination with the accompanying drawings and embodiments.

Unless otherwise defined, the technical or scientific terms used herein should have ordinary meanings understood by those of ordinary skill in the art of the present invention.

It is apparent to those skilled in the art that the present invention is not limited to the details of the above-mentioned exemplary embodiments and can be realized in other specific forms without departing from the intention or essential features of the present invention. Therefore, in all respects, the embodiments should be considered to be exemplary and non-restrictive. The scope of the present invention is limited by the appended claims rather than the above-mentioned description, so that all changes falling within the meaning and scope of the equivalents of the claims are intended to be included in the present invention, and any accompanying drawing marks in the claims should not be deemed to limit the claims involved.

Moreover, it should be understood that although the specification is described according to the implementation modes, not each implementation mode contains only one independent technical solution. This narrative form of the specification is for the sake of clarity only. Those skilled in the art should take the specification as a whole, and the technical solutions in various embodiments may be combined appropriately to form other implementation modes that can be understood by those skilled in the art. These other implementation modes should also fall within the protection scope of the present invention.

In addition, it should be understood that the above-mentioned specific embodiments are used for explaining the present invention only, and the protection scope of the present invention is not limited to such specific embodiments. Within the technical scope disclosed by the present invention, the equivalent substitutions or changes made by those skilled in the art based on the technical solutions and inventive concept of the present invention should fall within the protection scope of the present invention/invention.

The “including/comprising” or “containing” and similar words used herein refer to that the element ahead of the word covers the elements listed behind the word and does not exclude the possibility of covering other elements as well. The orientations or position relations indicated by terms “inside”, “outside”, “up” and “down” are those shown based on the accompanying drawings, only used for the convenience of describing the present invention and simplifying the description, rather than indicating or implying that the device or element referred to must have a particular orientation or be constructed and operated in a particular orientation, so they cannot be understood as a limitation to the present invention. When the absolute position of the object described changes, the relative position relation may also change accordingly. In the present invention, unless otherwise expressly specified and limited, the term “attaching” should be understood in a broad sense. For example, two elements may be connected fixedly, connected detachably, or integrated; two elements may be connected directly, or connected indirectly through an intermediate medium, or communicated internally or interact. Those of ordinary skill in the art can understand the specific meanings of such terms in the present invention according to the specific situations. The term “about” used herein has the meaning known to those skilled in the art, and preferably refers to that the value modified by the term is within the range of ±50%, ±40%, ±30%, ±20%, ±10%, ±5% or ±1%.

All terms (including technical or scientific terms) used in the disclosure have the same meanings as those understood by those of ordinary skill in the art of the disclosure, unless otherwise specifically defined. Moreover, it should be understood that terms defined in a general dictionary should be understood to have meanings consistent with those in the context of the relevant techniques, and should not be interpreted in an idealized or highly formal sense, unless expressly defined herein.

The techniques, methods and equipment known to those of ordinary skill in the art may not be discussed in detail, but where appropriate, such techniques, methods and equipment should be considered as a part of the specification.

The contents disclosed in the prior art literature referenced in the specification of the present invention are incorporated herein by reference in its entirety.

### Example 1

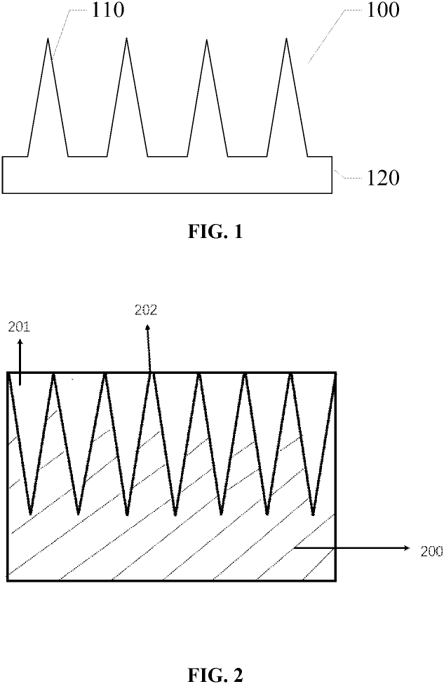

As shown in FIG. 1, the mRNA vaccine microneedle product 100 of the present invention includes a backing 120 and a mRNA vaccine microneedle array attached to a side of the backing 120, wherein the mRNA vaccine microneedle array includes several microneedles 110, wherein each microneedle 110 contains a matrix and a mRNA solution loaded in the matrix.

In the mRNA vaccine microneedle product 100, there is no special limitation to a matrix material forming a matrix, and all common matrix materials in the field can be applied in the present invention. However, considering that the microneedles 110 formed in the present invention need to have a certain mechanical strength, and the cured microneedles 110 need to have a certain porosity, the matrixes are preferably formed by crosslinking and/or drying and curing of an aqueous solution containing one or more of the following substances: polyethylene glycol diacrylate, silk fibroin, GelMA, carboxymethyl cellulose, trehalose, hyaluronic acid, polylactic acid-glycolic acid copolymer, polylactic acid, galactose, PVP and polyvinyl alcohol, preferably GelMA and/or hyaluronic acid, and more preferably GelMA.

Herein, the matrix material is preferably GelMA and/or hyaluronic acid. The GelMA is prepared from methacrylic anhydride (MA) and gelatin, which are crosslinked by UV light under the mediation of photosensitizer to form a pore structure with a certain strength. The GelMA features excellent biocompatibility. The hyaluronic acid is a mucopolysaccharide, which has a function of skin protection and can be applied to accelerate wound healing.

In the mRNA vaccine microneedle product 100, there is no special limitation to a backing material forming a backing 120, and all common backing materials in the field can be applied in the present invention. However, in the present invention, considering that the formed backing 120 needs to have a certain mechanical strength and flexibility, the backing 120 is preferably formed by crosslinking and/or drying of an aqueous solution containing one or more of the following substances: polyethylene glycol diacrylate, silk fibroin, GelMA, carboxymethyl cellulose, trehalose, hyaluronic acid, polylactic acid-glycolic acid copolymer, polylactic acid, galactose, PVP and polyvinyl alcohol, and preferably hyaluronic acid.

The hyaluronic acid is a mucopolysaccharide, which has a function of skin protection and can be applied to accelerate wound healing, so such hyaluronic acid is preferably used as a backing material in the present invention.

Herein, there is no special limitation to a thickness of the formed backing 120, but considering that the backing 120 needs to have a certain strength and flexibility, such thickness is preferably 0.1-15 mm, more preferably 1-10 mm, and most preferably 2-3 mm.

In addition, in some cases, in the mRNA vaccine microneedle product 100, materials forming the matrixes and the backing 120 are the same. In these cases, the microneedles 110 and the backing 120 are combined more stably, and in the following method for preparing the mRNA vaccine microneedle product 100, the microneedle array and the backing 120 can be integrated, simplifying a preparation process. In other cases, the materials of the matrixes and the backing 120 are preferably different; e.g., the matrix material is GelMA, while the backing material is hyaluronic acid. In such cases, the microneedles 110 have a certain strength, a pore structure and excellent biocompatibility; in addition, the backing 120 has a better protective effect on the skin, and can be applied to accelerate wound healing.

In addition, in the mRNA vaccine microneedle product 100, there is no special limitation to the size and shape of the microneedles 110, and both can vary over a wide range according to an application part of the mRNA vaccine microneedle product 100.

As shown in FIG. 1, in the mRNA vaccine microneedle product 100, each microneedle 110 includes a tip and a bottom, wherein the tip is away from the backing 120, each microneedle 110 is attached to the backing 120 via the bottom, and there is no special limitation to a height h between the tip and the bottom, but preferably 200 µm-1 mm. The height is preferably no lower than 200 µm, otherwise the microneedles 110 will not pierce some animal bodies, preferably the skin cuticles of some parts of a human body. However, the height is preferably no higher than 1 mm, otherwise the microneedles will pierce some animal bodies, preferably the skin cuticles of some parts of a human body, and reach the nervous layer, thus causing pain.

In addition, as shown in FIG. 1, in the mRNA vaccine microneedle product 100, the bottom of each of the microneedles 110 preferably has a diameter of 100 µm-500 µm. The diameter of the bottom is preferably no less than 100 µm, otherwise the microneedles 110 will be easy to break due to insufficient mechanical strength. In addition, the diameter of the bottom is preferably no more than 500 µm, otherwise after the mRNA vaccine microneedle product 100 is applied to some animal bodies, preferably some parts of a human body, larger holes will be left on the skin, leading to problems concerning skin beauty and healing.

In addition, in the mRNA vaccine microneedle product 100, there is no special limitation to a stereo shape of the microneedles 110, and the microneedles can be in a shape of cylinder, cone, circular truncated cone, etc., or a combination of them, and preferably regular or irregular circular cone, conoid, triangular pyramid, rectangular pyramid or higher-level pyramid which may be a right cone or an oblique cone.

In addition, as shown in FIG. 1, in the mRNA vaccine microneedle product 100, a spacing between the adjacent tips of the microneedles 110 is preferably 300 µm-800 µm. The spacing within such range can make the mRNA vaccine microneedle product 100 achieve an optimization effect in a depth of piercing the cuticle, etc.

It should be noted that those skilled in the art are capable of appropriately selecting shapes, sizes, etc. of the microneedles 110 in the mRNA vaccine microneedle product 100 according to practical application situations.

### Example 2

A method for preparing the mRNA vaccine microneedle product 100 includes the following steps:

S1, a mRNA solution was prepared: plasmids which contained designed sequence modules (including spike protein coding) were constructed first. Cell membranes were broken by electric current, and circular DNA plasmids were introduced into Escherichia coli. The Escherichia coli was stored in a solution containing a large amount of nutrients, and reproduction and amplification were performed to obtain trillions of DNA plasmids. The DNA plasmids were extracted and purified, and the solution was filtrated to remove bacteria and other substances. The purified circular DNA plasmids were cleaved into strands by enzyme. The DNA strands were transformed into mRNA. The DNA strands obtained in the previous step were mixed with enzyme and nucleotide, so that the RNA polymerase transcribed DNA into mRNA. The mRNA was encapsulated into a lipid carrier. The lipid was suspended in an alcoholic solution, came into contact with the mRNA and encapsulated the mRNA, and such two substances were attracted by opposite charges. Later, excess lipid, alcohol and other impurities were filtered from the stock solution through TFF, and a final mRNA vaccine solution was prepared.

S2, a matrix material forming a matrix was added to the mRNA-containing solution obtained in S1 to form a mixed solution.

In S2, there is no limitation to a concentration of the matrix material in the mixed solution, as long as an amount of the matrix material is sufficient to form microneedles 110 in a final mRNA vaccine microneedle product 100 of the present invention. However, a content of the matrix material in the liquid medium based on weight percentages is 20%-40%, preferably 25%-35%, and more preferably 30%. In such ranges, the microneedles 110 formed by the matrix material have sufficient mechanical strength and a certain porosity to achieve an optimum hydrogen generation efficiency.

S3, a microneedle mold 200 was provided. As shown in FIG. 2, the microneedle mold 200 includes an upper surface 202 and molding holes 201 extending downward from the upper surface 202, wherein each of the molding holes 201 includes a tip and a bottom, the tip is away from the upper surface 202, and the bottom plane is flush with the upper surface 202.

In the microneedle mold 200, a stereo shape of the molding holes 201 should be matched with the expected shape of the microneedles 110, and the molding holes can be in a shape of cylinder, cone, circular truncated cone, etc., or a combination of them, and preferably regular or irregular circular cone, conoid, triangular pyramid, rectangular pyramid or higher-level pyramid which may be a right cone or an oblique cone.

Each of the molding holes 201 should have a height, a bottom width and a tip spacing corresponding to those of the microneedles 110. However, in some cases, each of the molding holes 201 can further have a height and a bottom width larger than those of the microneedles 110. In the latter case, the formed microneedles 110 will not fill the whole space of the molding holes 201.

Optionally, the upper surface 202 (including inner surface of the molding holes 201) of the microneedle mold 200 can be coated with an anti-bonding layer.

In the present invention, the microneedle mold 200 is commercially available; for example, it can be a customized PDMS mold purchased from Taizhou Chipscreen Medical Technology Company, and the mold parameters can be customized according to the needs of the needle body size. Specifically, in the microneedle mold 200 adopted in the example of the present invention, the heights of all molding holes 201 were 600 µm, the bottom widths were 320 µm, the tip spacings were 500 µm, and the overall microneedle mold 200 was L × W=15 mm × 15 mm.

S4, the mixed solution obtained in S2 was placed in the molding holes 201 and filled at least a portion of the volume of the molding holes 201.

In S4, there is no limitation to the volume of the molding holes 201 filled by the mixed solution, but preferably at least ¼, ⅓, ½, ⅔ and ¾ of the volume of the molding holes 201 is filled by the mixed solution, and most preferably the whole volume is filled by the mixed solution. When the whole volume of the molding holes 201 is not filled, the portion of the molding holes 201 that is not filled by the microneedles 110 should be filled by a backing material solution in S6 below.

S5, the mixed solution containing the matrix material and the mRNA solution in the molding holes 201 was crosslinked and/or dried and cured to form microneedles 110 in the molding holes 201, and several microneedles 110 formed a mRNA vaccine microneedle array.

S6, a backing material-containing solution was applied to bottom surfaces of the microneedles 110 and the upper surface 202 of the microneedle mold 200 that was not covered by the microneedles 110 to form a backing solution layer, so that the mRNA vaccine microneedle array was attached to the backing solution layer.

In the mRNA vaccine microneedle product 100, the backing material is preferably silk fibroin, GelMA, hyaluronic acid, polylactic acid-glycolic acid copolymer, polylactic acid, galactose, polyvinyl alcohol or a mixture of two or more, and preferably hyaluronic acid.

S7, the backing solution layer obtained in S6 and optional microneedle array simultaneously attached to the backing solution layer were dried and cured to form a mRNA vaccine microneedle product 100.

In the drying step, a water content in the microneedles 110 and the backing 120 based on weight percentages is lower than 20%, and preferably lower than 5%.

In the method for preparing the mRNA vaccine microneedle product 100, the above-mentioned technical features and optimal ranges of the mRNA vaccine microneedle product 100 are still applicable herein.

### Test

The vaccine microneedle product and blank microneedle product were used for mouse test. The microneedle patches were applied to the skins of the points corresponding to the lungs of mice, and the mice were infected with the virus. The effects are as follows.

TABLE 1

Therapeutic Effects of Mice by Microneedles

| Group Effect | Control Group | | | | | Test Group | | | | |
|---|---|---|---|---|---|---|---|---|---|---|
| | 1 | 2 | 3 | 4 | 5 | 1 | 2 | 3 | 4 | 5 |
| IgG Log10 (ELISA EC50) | 0 | 0 | 0.7 | 0 | 0 | 1.5 | 1.6 | 1.4 | 1.3 | 1.5 |
| Body weight (g) of mice | 0 | 0 | 43 | 0 | 0 | 44 | 44 | 46 | 43 | 47 |
| Clinical score of mice | 0 | 0 | 6 | 0 | 0 | 7 | 7 | 6 | 7 | 7 |
| Survival result of mice | Dead | Dead | Alive | Dead | Dead | Alive | Alive | Alive | Alive | Alive |
| Survival rate of mice | 20% | | | | | 100% | | | | |

### Example 3

An application of the mRNA vaccine microneedle product prepared in example 1 and the method for preparing the mRNA vaccine microneedle product in example 2 in preparing medical devices and drugs against pneumonia.

The technical features and optimal ranges of such mRNA vaccine microneedle product and the preparation method thereof are still applicable herein.

Therefore, the present invention adopts the mRNA vaccine microneedle product as well as the preparation method and the application thereof. A vaccine present in the microneedle patch can not only work for a long time, but also reduce the discomfort caused by intramuscular injection and the use of medical resources.

Finally, it should be stated that the above-mentioned embodiments are only used for describing, rather than limiting, the technical solutions of the present invention. Although the present invention is described in detail by reference to the preferred embodiments, those of ordinary skill in the art should understand that they can still make modifications or equivalent substitutions to the technical solutions of the present invention, but these modifications or equivalent substitutions will not make the modified technical solutions deviate from the spirit and scope of the technical solutions of the present invention.

What is claimed is:

1. A mRNA vaccine microneedle product, comprising a backing and a mRNA vaccine solution-containing microneedle array attached to a side of the backing, wherein the mRNA vaccine solution-containing microneedle array comprises a plurality of microneedles, wherein each of the plurality of microneedles comprises a matrix and mRNA loaded in the matrix.

2. The mRNA vaccine microneedle product of claim 1, wherein each of the plurality of microneedles comprises a tip and a bottom, wherein the tip is away from the backing, the bottom is attached to the backing, a vertical distance from the tip to the bottom is 200 µm-1 mm, a diameter of the bottom is 100 µm-500 µm, and a spacing between the adjacent tips is 300 µm-800 µm.

3. The mRNA vaccine microneedle product of claim 1, wherein a material of the matrix is at least one compound selected from the group consisting of polyethylene glycol diacrylate, silk fibroin, gelatin, hyaluronic acid, polylactic acid-glycolic acid copolymer, polylactic acid, PVP, and polyvinyl alcohol, preferably gelatin and/or hyaluronic acid, and more preferably gelatin, and
the matrix is formed by crosslinking and/or drying and curing of an aqueous solution of the compound.

4. The mRNA vaccine microneedle product of claim 1, wherein a material of the backing is at least one compound selected from the group consisting of polyethylene glycol diacrylate, silk fibroin, GelMA, carboxymethyl cellulose, trehalose, hyaluronic acid, polylactic acid-glycolic acid copolymer, polylactic acid, galactose, PVP, and polyvinyl alcohol, and
the backing is formed by crosslinking and/or drying and curing of an aqueous solution of the compound.

5. A preparation method of the mRNA vaccine microneedle product according to claim 1, comprising the following steps:
1) preparing an mRNA vaccine-containing liquid medium;
2) adding a matrix material forming the matrix to the mRNA vaccine-containing liquid medium obtained in step 1 to form a mixed solution;
3) placing the mixed solution obtained in step 2 in molding holes of a microneedle mold, and filling at least a portion of a volume of the molding holes, and preferably a whole volume of the molding holes;
4) crosslinking the mixed solution containing the matrix material and the mRNA vaccine-containing liquid medium in the molding holes, and preferably performing ultraviolet crosslinking for 10s, and/or performing drying and curing, and preferably performing hot curing, to form the plurality of microneedles in the molding holes;
5) applying a solution containing a material of the backing to bottom surfaces of the plurality of microneedles and an upper surface of the microneedle mold that is not covered by the plurality of microneedles to form a backing solution layer, and crosslinking the backing material to form a continuous backing layer, so that the mRNA vaccine solution-containing microneedle array is attached to a backing solution layer or the continuous backing layer; and
6) drying and curing the backing solution layer or the continuous backing layer obtained in step 5 and the mRNA vaccine solution-containing microneedle array to form the mRNA vaccine microneedle product.

6. The preparation method of claim 5, wherein step 1 for preparing the mRNA vaccine-containing liquid medium comprises the following steps:
a) breaking cell membranes of Escherichia coli by electric current, and introducing circular DNA plasmids into the Escherichia coli;
b) storing the Escherichia coli in a solution containing a large amount of nutrients and performing reproduction and amplification to obtain trillions of DNA plasmids;
c) extracting and purifying the DNA plasmids, and filtrating a solution to remove bacteria and other substances and obtain purified circular DNA plasmids;
d) cleaving the purified circular DNA plasmids into DNA strands by an enzyme;
e) transforming the DNA strands into mRNA: mixing the DNA strands obtained in the previous step with enzymes and nucleotides, so that an RNA polymerase transcribes the DNA strands into the mRNA;
f) encapsulating the mRNA into a lipid carrier to obtain a stock solution, wherein the lipid carrier is suspended in an alcoholic solution, the lipid carrier comes into contact with the mRNA and encapsulates the mRNA, and the lipid carrier and the mRNA are attracted by opposite charges; and
g) filtering excess impurities from the stock solution through tangential flow filtration (TFF), and preparing the mRNA vaccine-containing liquid medium.

7. The preparation method of claim 6, wherein in step 2, a concentration of the matrix material in the mixed solution is 20%-40% of a total weight of the mixed solution, and preferably 30%.

8. The preparation method of claim 6,
wherein in step 2, the microneedle mold is provided, and the microneedle mold comprises an upper surface and the molding holes extending downward from the upper surface,
wherein each of the molding holes comprise a tip and a bottom, the tip is away from the upper surface, a plane of the bottom is flush with the upper surface, a distance from the tip to the bottom is 200 µm-1 mm, a diameter of the bottom is 100 µm-500 µm, and a spacing between the adjacent tips is 300 µm-800 µm.

9. The preparation method of claim 6, wherein the backing material is hyaluronic acid, and the matrix material is methacrylate gelatin (GelMA).

10. An application of the mRNA vaccine microneedle product according to claim 1 in preparing medical devices or drugs against pneumonia-associated viruses.

11. An application of the preparation method of the mRNA vaccine microneedle product according to claim 5 in preparing medical devices or drugs against pneumonia-associated viruses.
